(19) United States
(12) Patent Application Publication
(10) Pub. No.: US 2010/0137333 A1
(43) Pub. Date: Jun. 3, 2010

(54) ANTIFUNGAL COMPOSITION AND METHODS FOR USING

(76) Inventor: James A. Roszell, Henderson, NV (US)

Correspondence Address: MERCHANT & GOULD PC, P.O. BOX 2903, MINNEAPOLIS, MN, 55402-0903, US

(21) Appl. No.: 12/445,819

(22) PCT Filed: Oct. 19, 2007
(86) PCT No.: PCT/US2007/022327

§ 371 Date: Jan. 28, 2010

Related U.S. Application Data
(60) Provisional application No. 60/853,073, filed on Oct. 20, 2006.

(51) Int. Cl. A61K 31/496; A61K 31/4164; A61K 31/27; A61K 31/137; A61K 31/4196; A61P 31/10

(52) U.S. Cl. 514/254.07; 514/396; 514/481; 514/655; 514/399; 514/398; 514/383; 514/654

(57) ABSTRACT

An antifungal composition is provided according to the present invention. The antifungal composition comprises an effective amount of an antifungal component to provide antifungal properties to skin tissue, an effective amount of a skin bonding polymer component to provide a sustained release of the antifungal component over a prolonged period of time after application of the antifungal treatment composition to skin tissue, and water. A method for using the antifungal composition is provided.

This application is being filed on 19 Oct. 2007, as a PCT International Patent application in the name of Skinvisible Pharmaceuticals, Inc., a U.S. national corporation, applicant for the designation of all countries except the US, and James A. Roszell, a citizen of the U.S., applicant for the designation of the US only, and claims priority to U.S. Provisional Patent Application Ser. No. 60/853,073, filed Oct. 20, 2006.

### FIELD OF THE INVENTION

The invention relates to a topical antifungal composition and to methods for using a topical antifungal composition. In particular, the antifungal composition can include an effective amount of an antifungal active component and an effective amount of a skin bonding polymer component, and can provide a sustained release of the antifungal component over a prolonged period of time.

### BACKGROUND OF THE INVENTION

Pharmaceutical compositions are available for the treatment of fungal infections. Pharmaceutical compositions can be provided as topical compositions for addressing fungal infections in skin tissue and in nails such as toenails. Exemplary disclosures of topical compositions for treating fungal infections include U.S. Pat. No. 6,080,744 to Ayon-Covarrubias, U.S. Pat. No. 6,077,865 to Som, U.S. Pat. No. 4,803,066 to Edwards, and U.S. Publication Number US 2003/0235541 to Maibach et al.

There is a desire to provide a topical, antifungal treatment composition that provides a sustained release of an antifungal active amount of an antifungal component over a prolonged period of time.

### SUMMARY OF THE INVENTION

An antifungal composition is provided according to the present invention. The antifungal composition comprises an effective amount of an antifungal component to provide antifungal properties to skin tissue, an effective amount of a skin bonding polymer component to provide a sustained release of the antifungal component over a prolonged period of time after application of the antifungal treatment composition to skin tissue, and water.

Exemplary antifungal components include clotrimazole, naftifine, ketoconazole, tolnaftate, terbinafine, butenafine, miconazole, metronidazole, fluconazole, and mixtures thereof. The antifungal composition can include about 0.1 wt. % to about 5 wt. % of the antifungal component.

A method for using an antifungal composition is provided according to the present invention. The method includes a step of applying the antifungal composition to skin tissue. The method provides for a sustained release of the antifungal component over a prolonged period of time. A prolonged period of time can be a time of at least about one hour. A sustained release of the antifungal component can refer to a release sufficient to provide antifungal properties over the time period.

### BRIEF DESCRIPTION OF THE DRAWING

FIG. 1 is a graph showing the result of Example 1.

FIG. 2 is a graph showing the result of Example 2.

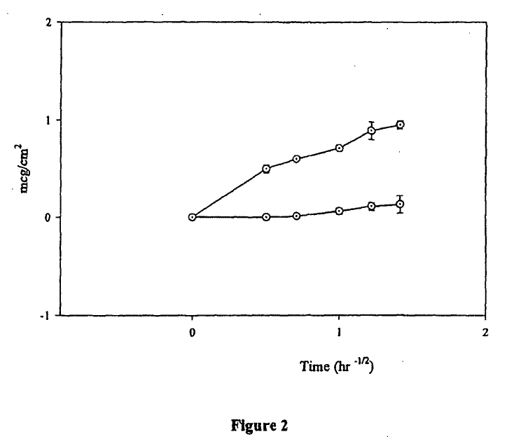

### DETAILED DESCRIPTION OF THE INVENTION

An antifungal composition is provided that exhibits antifungal properties when applied topically to tissue such as skin tissue. Prolonged antifungal properties refers to antifungal properties that persist over a period of time. In general, the persistence can be considered sufficient so that the tissue having the antifungal composition applied thereto exhibits antifungal properties for at least one hour after application of the composition to tissue. The antifungal composition preferably provides antifungal properties at least about two hours after application to tissue and preferably at least about four hours after application to tissue.

The antifungal composition can be applied typically to tissue on virtually any part of the body to provide disinfectant properties. Exemplary tissue includes skin tissue and nails such as toenails. The antifungal composition can be applied to various areas to treat ringworm, athlete's foot, jock itch, etc.

The antifungal composition can be provided in the form of a lotion and applied to skin tissue by rubbing the composition onto the skin tissue. The antifungal composition can have a viscosity that allows it to be applied to skin tissue conveniently as a lotion. The antifungal composition can have a viscosity that is sufficiently high so that the lotion can be applied from a container (e.g., a tube or a bottle) to a person's hand or a location on the person's body, and the lotion can be rubbed onto the skin tissue. When provided as a lotion, the antifungal composition can have a viscosity of greater than about 3,000 cSt (centistokes). When provided as a lotion, the antifungal composition can be referred to as an antifungal lotion or more simply as a lotion. The antifungal composition can be provided in a form having a viscosity of less than about 3,000 cSt. When the antifungal composition is provided having a viscosity of less than about 3,000 cSt, the antifungal composition can be called an antifungal liquid or more simply as a liquid.

The antifungal composition includes an antifungal component, a skin bonding polymer component, and water. Additional components can include surfactant, pH modifying agent, coloring agent, preservative, thickening agent, emollient, humectant, antioxidant, fragrance, and chelating agent. The antifungal composition can include any one or more of these additional components.

The antifungal composition can be provided as an emulsion. Exemplary types of emulsions include oil in water emulsions, and water in oil in water emulsions.

The terms “antifungal component” and “antifungal agent” are used herein interchangeably to refer to a pharmaceutically acceptable agent that, when administered to a human patient as part of a topical composition provides a desired pharmacologic and/or physiologic effect by local and/or systemic action. Also included are derivatives and analogs of those compounds or classes of compounds specifically mentioned that also induce the desired effect.

The term “pharmaceutically acceptable” is meant to characterize a compound that is not biologically or otherwise undesirable, i.e., the compound may be incorporated into a topical formulation of the invention and administered to a patient without causing any undesirable biological effects or interacting in a deleterious manner with any of the other components of the formulation in which it is contained. A “pharmacologically active” compound refers to an active agent or to an analog or derivative thereof having the same type of pharmacological activity as the parent compound.

The terms “treating” and “treatment” as used herein refer to actions that reduce the severity and/or frequency of symptoms, eliminate symptoms and/or their underlying cause, prevent the occurrence of symptoms and/or their underlying cause, and improve or remediate damage. Treating a patient, as the term is used herein, thus encompasses both prevention and treatment.

### Skin Bonding Polymer Component

The antifungal composition can include a skin bonding polymer component. The skin bonding polymer component can include any polymer that, when applied to the skin, helps hold the antifungal component to the skin. The skin bonding polymer component holds the antifungal component to the skin tissue for a sufficient length of time to provide a desired disinfectant property. The skin bonding polymer component can be referred to as the polymer component. The polymer component can be characterized as a polymer having an average molecular weight of at least about 2,000, and as a polymer having an average molecular weight of less than about 500,000.

The polymer component can include a hydrophobic polymer/hydrophilic polymer adduct and can include other components. Polymer components that can be used according to the invention include the topical compositions disclosed in U.S. Pat. No. 6,756,059. The entire disclosure of U.S. Pat. No. 6,756,059 is incorporated herein by reference.

The antifungal composition can bind or adhere to skin tissue for a length of time, and can hold or contain the antifungal component within the composition. It is expected that the antifungal composition is able to adhere or bind to skin tissue for at least about one hour and preferably at least about two hours and hold the antifungal component contained therein in proximity to skin tissue for that length of time. In general, it is expected that the antifungal composition will adhere the antifungal component to skin tissue for a length of time sufficient to provide desired antifungal properties.

The polymer component can be prepared from a topical composition precursor. The topical composition precursor can be prepared by melt processing a hydrophobic polymer composition and a hydrophilic polymer composition to provide an interaction between the hydrophobic polymer composition and the hydrophilic polymer composition. It should be understood that the phrase “melt processing” refers to mixing the hydrophobic polymer composition and the hydrophilic polymer composition under conditions that provide that the hydrophobic polymer component of the hydrophobic polymer composition and the hydrophilic polymer component of the hydrophilic polymer composition are in a liquid state so that they sufficiently mix. When the polymers are sufficiently mixed, it is believed that an interaction forms between the hydrophobic polymer component and the hydrophilic polymer component. The melt processing temperature can be at least about 50° C. and can be at least about 70° C. to generate this interaction.

It is believed the interaction exhibited between the hydrophobic polymer component and the hydrophilic polymer component is a type of complex formation reaction, and that the complex, once formed, can be stable in water at temperatures up to 65° C. and at a pH range of 3.0 to 9.0. By stable, it is meant that the complex does not favor disassociation under these conditions. It is believed that this interaction provides the antifungal composition with an ability to bind or hold onto the disinfectant active component that may be hydrophobic or relatively water insoluble, allows the antifungal composition to be emulsified in water, and provides the antifungal composition with an ability to bind to skin. The result of the interaction between the hydrophobic polymer component and the hydrophilic polymer component can be referred to as a hydrophobic polymer/hydrophilic polymer adduct. It should be understood that the term “adduct” is used to refer to the interaction between the hydrophobic polymer component and the hydrophilic polymer component. The interaction may be a form of complexing, but that is only theory. Accordingly, it should be understood that the term “adduct” is not meant to limit the polymer component to a particular theory of interaction.

It is believed that the interaction between the hydrophobic polymer component and the hydrophilic polymer component can be achieved more easily in the absence of water. It is expected that that if the hydrophilic polymer component becomes dissolved in water before forming the complex, it can be more difficult to sufficiently mix the hydrophobic polymer component and the hydrophilic polymer component to provide the desired level of interaction. Although a convenient technique for providing the desired level of interaction between the hydrophobic polymer component and the hydrophilic polymer component is melt mixing, it is expected that other techniques can be used to achieve the desired level of interaction. For example, it may be possible to use a nonaqueous solvent to help achieve the desired level of interaction.

The hydrophobic polymer composition that can be used according to the invention includes at least one hydrophobic polymer and can include a mixture of hydrophobic polymers. The hydrophobic polymer composition can include components having repeating pyrrolidone/alkylene groups. Exemplary polymers having repeating pyrrolidone/alkylene groups include poly(vinylpyrrolidone/alkylene) polymers. Poly(vinylpyrrolidone/alkylene) polymers include those polymers obtained by polymerizing alkylene substituted vinylpyrrolidone. Poly(vinylpyrrolidone/alkylene) polymers can be represented by the following general formula:

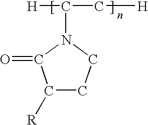

wherein R represents a carbon chain substitute such as an alkylene group and n represents the number of repeating units. The R group is preferably sufficiently long so that the polymer remains relatively water insoluble and should not be too long so that the polymer is difficult to melt process. The alkylene group can contain a length of at least about 10 carbon atoms and can contain less than about 30 carbon atoms. The alkylene group can contain about 14 carbon atoms to about 22 carbon atoms, and can contain about 15 carbon atoms to about 19 carbon atoms.

The poly(vinylpyrrolidone/alkylene) polymers that can be used according to the invention can have a molecular weight that is sufficiently high so that the polymer maintains its water insolubility but the molecular weight should not be so high that it becomes difficult to melt process the polymer. The weight average molecular weight of the poly(vinylpyrrolidone/alkylene) polymer can be between about 3,000 and about 400,000. Another way to characterize the size of the poly(vinylpyrrolidone/alkylene) polymer is by the number of repeating units (n). In the case of a poly(vinylpyrrolidone/alkylene) polymer having a weight average molecular weight of about 6,000 to about 30,000, the poly(vinylpyrrolidone/alkylene) polymer can have about 20 to about 80 repeating units, and can have about 30 to about 50 repeating units. It should be understood that repeating units refer to the residues of vinylpyrrolidone/alkylene groups.

Exemplary poly(vinylpyrrolidone/alkylene) polymers that can be used according to the invention include poly(vinylpyrrolidone/1-eicosene) and poly(vinylpyrrolidone/hexadecene). Poly(vinylpyrrolidone/1-eicosene) can be referred to as PVPE and is commonly used in pharmaceutical and cosmetic preparations. An exemplary form of PVPE for use according to the invention includes about 43 to 44 repeating units in length and has a weight average molecular weight of about 17,000 and can be characterized as a paraffin-like solid. This particular PVPE is highly insoluble in water, and has an extremely low oral toxicity (LD₅₀>17000 mg/kg) and exhibits no demonstrable dermal toxicity. Poly(vinylpyrrolidone/1-hexadecene) can be referred to as PVPH. An exemplary form of PVPH is available as a viscous yellow liquid that is insoluble in water and has a low oral toxicity (LD₅₀>64000 mg/kg), has about 39 to 40 repeating units, a molecular weight of about 14,000, and exhibits no demonstrable dermal toxicity.

PVPE and PVPH differ in the length of the hydrocarbon side chain, and are used extensively in the skin care industry, usually in concentrations of less than 1% by weight, because of their ability to bind to skin. Because the skin care industry generally prefers to apply actives to skin using a water-based composition, the use of PVPE and PVPH often requires solvents, surfactants, and emulsifiers to stabilize these polymers in a water emulsion. However, many of the solvents, surfactants and emulsifiers used to stabilize PVPE and PVPH in a water emulsion lack the low dermal toxicities of PVPE and PVPH. PVPE and PVPH by themselves lack a cosmetically elegant appeal when applied directly to the skin. They tend to be sticky and greasy.

The hydrophobic polymer composition used according to the invention can be provided as a mixture of different poly(vinylpyrrolidone/alkylene) polymers. The mixture of different poly(vinylpyrrolidone/alkylene) polymers can include at least 5 wt. % of a first poly(vinylpyrrolidone/alkylene) polymer based on the weight of the hydrophobic polymer composition. The hydrophobic polymer composition can include about 5 wt. % to about 54 wt. % of the first poly(vinylpyrrolidone/alkylene) polymer. The second poly(vinylpyrrolidone/alkylene) polymer can be provided in an amount of at least about 46 wt. % and can be in a range of about 46 wt. % to 95 wt. % based on the weight of the hydrophobic polymer composition. For a hydrophobic polymer composition containing a first poly(vinylpyrrolidone/alkylene) polymer and a second poly(vinylpyrrolidone/alkylene) polymer, the mole ratio of the first polymer to the second polymer can be about 1:22 to about 1:1. When the hydrophobic polymer composition contains a mixture of different poly(vinylpyrrolidone/alkylene) polymers, the poly(vinylpyrrolidone/alkylene) polymers can be selected to provide improved properties compared to a composition having a hydrophobic polymer composition containing a single poly(vinylpyrrolidone/alkylene) polymer.

When the hydrophobic polymer composition is provided as a mixture of PVPH and PVPE, the PVPH can be provided in a range of about 46 wt. % to about 95 wt. % and the PVPE can be provided in a range of about 5 wt. % to about 65 wt. %, based upon the weight of the hydrophobic polymer composition.

The hydrophilic polymer composition that can be used according to the invention includes at least one hydrophilic polymer and may include a mixture of hydrophilic polymers. The hydrophilic polymers that can be used according to the invention include polymers having repeating carboxylic acid groups, hydroxyl groups, or both carboxylic acid groups and hydroxyl groups. Exemplary hydrophilic polymers that can be used according to the invention include polyacrylic acid polymers, poly(maleic acid/methylvinylether) copolymers, starch, derivatives of starch, polyvinyl alcohol, cellulose, derivatives of cellulous, carboxymethyl cellulous, cyclodextrins, dextrans, or mixtures thereof. The hydrophilic polymers should have a molecular weight that is not too high so that the hydrophilic polymer becomes difficult to process.

Polyacrylic acid polymers that can be used according to the invention include those having a weight average molecular weight of at least about 50,000. Polyacrylic acid polymers that can be used include those having a weight average molecular weight between about 50,000 to about 4,000,000. The polyacrylic acid polymers can have a level of cross-linking that is less than about 1% to help provide hydrophilic properties. A general structural representation of polyacrylic acid polymers is shown below:

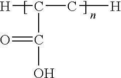

wherein n is the number of repeating units. The number n can be about 1,000 to about 20,000.

Poly(maleic acid/methylvinylether) copolymers that can be used according to the invention can have a weight average molecular weight of at least about 50,000, and can have a weight average molecular weight of about 50,000 to about 4,000,000. The weight average molecular weight can be about 70,000 to 2,500,000. A general structural representation of poly(maleic acid/methylvinylether) copolymers is shown below:

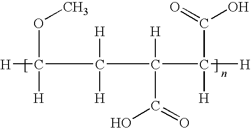

wherein n is the number of repeating units. The number n can be about 200 to about 20,000.

Additional hydrophilic polymers that can be used according to the invention include starch, derivatives of starch, polyvinyl alcohol, cellulose, derivatives of cellulose, carboxymethyl cellulose, cyclodextrins, and dextrans. The weight average molecular weight of the hydrophilic polymers is preferably sufficient to provide solubility in water but not too high to become difficult to process. Exemplary starches include amylopectin and polyglucose. Starches that can be used according to the invention can have a weight average molecular weight of about 50,000 to about 20,000,000. An exemplary starch component that can be used includes Zea Mays starch. A derivative of starch that can be used according to the invention includes partially hydrolyzed starch. Cellulose that can be used according to the invention can have a weight average molecular weight of about 50,000 to about 15,000,000. An exemplary cellulose compound that can be used includes cellulose gum. Polyglucose that can be used according to the invention can be characterized as low fraction polyglucose having a weight average molecular weight of about 60,000 to about 90,000, and high fraction polyglucose having a weight average molecular weight of about 90,000 to about 300,000. An exemplary low fraction polyglucose material that can be used according to the invention is available under the name Dextran-70. In general, this type of polyglucose has all alpha 1-6 linkages. Starch derivatives that can be used according to the invention include those starch derivatives having alpha 1-4 linkages. An example of this type of starch derivative includes cyclodextrins. Exemplary cyclodextrins that can be used according to the invention include those that act to provide a cavity within the molecule large enough to contain components desirable for topical applications. Cyclodextrins that can be used according to the invention can have a molecular weight of about 900 to about 1,400. Polyvinyl alcohols that can be used according to the invention include those with a weight average molecular weight of about 50,000 to about 200,000.

Exemplary hydrophilic polymers that can be used according to the invention include those polymers having a melting temperature that allows for melt processing without decomposition of the polymer. Exemplary poly(maleic acid/methylvinylether) copolymers that can be used include those having a melting temperature range of about 60° C. to about 65° C. and a maximum temperature range of about 80° C. to about 90° C. The melting temperature refers to the temperature at which the polymer melts, and the maximum temperature refers to the temperature at which the polymer begins to decompose. Exemplary polyacrylic acid polymers that can be used include those having a melting temperature range of about 65° C. to about 70° C. and a maximum temperature range of about 80° C. to about 90° C. Exemplary carboxymethyl cellulose polymers that can be used include those having a melting temperature range of about 55° C. to about 60° C. and a maximum temperature range of about 75° C. to about 80° C. Exemplary polyvinyl alcohol polymers that can be used include those having a melting temperature range of about 50° C. to about 55° C. and a maximum temperature range of about 65° C. to about 70° C. Exemplary starches that can be used include those having a melting temperature range of about 40° C. to about 45° C. and a maximum temperature range of about 50° C. to about 55° C. Exemplary dextrans that can be used include those having a melting temperature range of about 37° C. to about 40° C. and a maximum temperature range of about 45° C. to about 50° C. Exemplary β-cyclodextrins that can be used according to the invention include those having a melting temperature range of about 40° C. to about 45° C. and a maximum temperature range of about 65° C. to about 70° C.

The hydrophobic polymer composition and the hydrophilic polymer composition can be combined and heated to at least about 50° C. to provide a polymer melt. The composition can be heated to at least about 70° C. under mixing to form complexes between the hydrophobic and hydrophilic polymers. It should be understood that a polymer melt refers to a polymer that flows or becomes a liquid when heated and is not meant to refer to a polymer that forms a liquid as a result of being dissolved in a solvent.

The complex formation step can be carried out in a relatively anhydrous environment. That is, the amount of water provided in the composition during the complex formation step can be less than about 1 wt. %. Once the desired level of complex formation has occurred, the composition can be hydrated with water.

The hydrophobic polymer composition and the hydrophilic polymer composition can be mixed together in amounts sufficient to provide a ratio of pyrrolidone groups to the combination of carboxylic acid groups and hydroxyl groups of about 1:1 to about 5:1. The ratio of the structures causing the observed interaction between the hydrophobic polymer composition and the hydrophilic polymer composition can be referred to as “functional group parity.” The ratio of pyrrolidone groups to the combination of carboxylic acid groups and hydroxyl groups can be about 1.5:1 to about 3:1. In order to drive the complex formation reaction, it is desirable to provide an imbalance between the two types of groups. Accordingly, it is generally desirable to provide more of the pyrrolidone groups than the combination of carboxylic groups and the hydroxyl groups. It should be understood that the reference to a “combination of carboxylic groups and hydroxyl groups” refers to the total amount of carboxylic groups and hydroxyl groups present but does not require the presence of both carboxylic groups and hydroxyl groups. For example, the value of the combination of carboxylic groups and hydroxyl groups can be determined for a composition that contains only carboxylic groups. Similarly, the value can be determined for a composition that contains only hydroxyl groups.

During the complex formation step, the amounts of hydrophobic polymer composition and hydrophilic polymer composition can be characterized on a weight percent basis. For example, about 2 wt. % to about 28 wt. % hydrophilic polymer composition and about 72 wt. % to about 98 wt. % hydrophobic polymer composition can be combined to provide for complex formation. About 8 wt. % to about 25 wt. % hydrophilic polymer composition and about 72 wt. % to about 95 wt. % hydrophobic polymer composition can be combined to form the complex. During the complex formation step, the amount of water available in the composition can be less than about 1 wt. %. Although the complex forming composition can be relatively anhydrous, it is expected that the amount of water will be between about 0.3 wt. % and about 1.0 wt. %.

Once the hydrophobic polymers and the hydrophilic polymers have sufficiently reacted or interacted to form a complex, water can be added to the composition to provide a stable aqueous composition that can be relatively easily further hydrated. It has been found that the first hydration of the topical composition precursor is the most difficult hydration step because of the need to control the conditions of hydration. After the first hydration to a water content of at least about 30 wt. %, it is expected that further hydrations to higher water contents are relatively easy and can be accomplished by simply mixing the composition with water. Accordingly, the amount of water provided in the composition when made available as a concentrate for shipment is preferably between about 30 wt. % and about 45 wt. %. When the composition includes about 30 wt. % to about 45 wt. % water, it is expected that the composition can include about 3 wt. % to about 10 wt. % hydrophilic polymer composition and about 30 wt. % to about 50 wt. % hydrophobic polymer composition.

Water can be added to the relatively anhydrous composition by mixing water and the relatively anhydrous composition at a temperature and for a time sufficient to allow the composition to become hydrated without losing significant amounts of interaction between the hydrophobic polymer composition and the hydrophilic polymer composition. The relatively anhydrous composition can be hydrated by heating to at least 60° C. and adding water while mixing. The composition can be heated to at least about 65° C. and to at least about 70° C. An exemplary temperature range is about 65° C. to about 80° C.

The relatively anhydrous composition can be referred to as the topical composition precursor and generally refers to the hydrophobic polymer/hydrophilic polymer adduct. The polymer component for the hand disinfecting composition can refer to a composition that contains only the hydrophobic polymer/hydrophilic polymer adduct, and it can refer to a composition wherein the hydrophobic polymer/hydrophilic polymer adduct is diluted with water. In general, it is desirable to have a sufficient amount of water in the polymer component that allows one to formulate the polymer component into the antifungal composition according to the invention. If there is too little water in the polymer component, it may become difficult to formulate the antifungal composition. For example, the polymer component can contain water in an amount of up to about 95 wt. %. The polymer component can have a water concentration of about 30 wt. % to about 45 wt. %.

Additional components can be added to the skin bonding polymer composition. For example, it may be desirable to add a component that helps stabilize the hydrophobic polymer/hydrophilic polymer adduct, and to help preserve and/or maintain the composition.

The antifungal treatment composition can include the skin bonding polymer component in an amount sufficient to provide desired bonding properties of the composition. For example, the antifungal treatment composition can include about 3 wt. % to about 20 wt. % of the skin bonding polymer component, about 4 wt. % to about 15 wt. % of the skin bonding polymer component, or about 5 wt. % to about 8 wt. % of the skin bonding polymer component.

### Antifungal Component

The antifungal composition includes an antifungal component or an antifungal agent that provides antifungal active properties. The antifungal composition can include any antifungal component that is compatible with the skin bonding polymer component and provides desired disinfectant properties. In general, compatibility of the antifungal component with the skin bonding polymer component refers to the lack of phase separation between the antifungal component and the skin bonding polymer component during storage. It is generally desirable for the skin bonding polymer component to hold the antifungal component and, when applied to tissue, to release a desired amount of the antifungal component to the tissue to provide a desired level of activity over a desired length of time. A desired level of activity refers to a level that provides a pharmaceutically active effect.

The antifungal composition can be provided so that it remains on skin tissue for a prolonged period of time and provides a sustained release of the antifungal component over the prolonged period of time. In general, a prolonged period of time refers to a time of at least about one hour, preferably at least about two hours, and more preferably at least about four hours. A sustained release of the antifungal component refers to a release of the antifungal component over the stated period wherein the antifungal component continues to provide antifungal activity. For example, for an antifungal composition having a sustained release for one hour, the composition provides antifungal activity for one hour. For an antifungal composition providing sustained release for two hours, the composition provides antifungal properties for two hours. Furthermore, for an antifungal composition providing a sustained release of four hours, the composition provides antifungal activity for four hours. If desired, the sustained release can be further characterized by providing a rate of release of the antifungal component at one hour that is within about 50% of the rate of release of the antifungal component at 30 minutes. Furthermore, the composition can be provided so that it provides a rate of release of the antifungal component at two hours that is within about 50% of the release rate of the antifungal component at 30 minutes. Preferably, the rate of release can be within about 25%, and more preferably can be within about 15%. There are a couple advantages associated with providing an antifungal composition exhibiting sustained release. By providing for a sustained release of the antifungal component, it is possible to prolong the activity of the composition compared with a composition that essentially releases all of the antifungal component at once. Accordingly, by providing for a sustained release of the antifungal component, the composition can be applied to tissue less often and provide a more effective treatment. Furthermore, certain antifungal components have a tendency to cause irritation to tissue when applied at certain levels. By controlling the rate of release of the antifungal component, and thereby controlling the concentration of the antifungal component released, it is possible to reduce the tendency of the antifungal component to cause skin irritation.

Antifungal agents include, without limitation: amorolfine; ciclopirox olamine; flucytosine; griseofulvin; haloprogrin; potassium iodide sodium pyrithione; undecylenic acid; imidazole derivatives, including without limitation bifonazole, butoconazole, clotrimazole, econazole, ketoconazole, miconazole, oxiconazole, and sulconazole; triazoles, including without limitation itraconazole, fluconazole, and terconazole; allylamines, including without limitation naftifine, butenafine, and terbinafine; polyene antifungal antibiotics such as amphotericin B and nystatin; antifungal organic acids such as benzoic acid, salicylic acid, propionic acid, and caprylic acid; and a propylene glycol-urea-lactic acid combination. One or more additional active antifungal agents may optionally be present in the formulation. Preferred antifungal agents include clotrimazole, naftifine, ketoconazole, tolnaftate, terbinafine, butenafine, and miconazole.

In general, the antifungal composition can include the antifungal component in an amount of about 0.1 wt. % to about 3 wt. %. For specific antifungal components, the amount of the antifungal component can be provided at a particular range. For example, clotrimazole can be provided in an amount of about 0.5 wt. % to about 1.5 wt. %, naftifine can be provided in an amount of about 0.5 wt. % to about 1.5 wt. %, ketoconazole can be provided in an amount of about 0.5 wt. % to about 1.5 wt. %, tolnaftate can be provided in an amount of about 0.5 wt. % to about 1.5 wt. %, terbinafine can be provided in an amount of about 0.5 wt. % to about 1.5 wt. %, butenafine can be provided in an amount of about 0.5 wt. % to about 1.5 wt. %, and miconazole can be provided in an amount of about 1.0 wt. % to about 3.0 wt. %.

### Water

The antifungal composition can include water in an amount sufficient to allow the composition to be applied to skin tissue while providing the desired coverage over the skin tissue. The water component can be provided as deionized water, filtered water, distilled water, reverse osmosis water, or tap water. In the event that the water includes hardness or other components, it may be desirable to include builders, sequestrants, and chelating agents to handle the water hardness. In general, the hand antifungal composition can include at least about 50 wt. % water. In addition, it is expected that if there is too much water, the emulsion might become unstable. In general, the amount of water in the antifungal composition can be less than about 95 wt. %. The amount of water in the antifungal composition can be about 65 wt. % to about 93 wt. %.

### Release Component

The antifungal composition can include a release agent to assist with the sustained release of the antifungal component over a prolonged period of time. The release agent can be provided as a surfactant. A surfactant can additionally be present to help maintain the antifungal treatment composition as an emulsion. In general, an emulsion refers to a composition that resists phase separation after sitting at room temperature for a couple of months. In general, it is expected that the antifungal composition can be stored in a warehouse or in a storage closet for at least two months and can remain as an emulsion during that two month period. Preferably, the antifungal composition can remain as an emulsion for at least one year or at least two years. The ability of the antifungal composition to remain as an emulsion can be tested according to an accelerated stability test where the composition is held at 45° C. for two months. It is expected that this accelerated stability test for two months roughly corresponds to a period of about two years at room temperature. In general, it is expected that the hand disinfecting composition can remain as an emulsion after sitting for one month at 45° C. and preferably at least two months at 45° C.

Exemplary surfactants that can be used as the surfactant component include nonionic surfactants that help stabilize the emulsion and provide a generally even distribution of the antifungal component. Exemplary nonionic surfactants that can be used include glycerol stearate such as glycerol monostearate, polysorbate such as that available under the name Tween 80, and polyoxyethylene stearate. In addition, mixtures of nonionic surfactants can be included including mixtures of polysorbate and glycerol stearate. An additional nonionic surfactant that can be used includes an ethoxy surfactant, a propoxy surfactant, or an ethoxy/propoxy surfactant. An exemplary ethoxy/propoxy surfactant includes a 10 carbon chain and 9 PO/EO surfactant available under the name Lutensol XP-90 from BASF. Additional nonanionic surfactants include sorbitan monolaurate and sorbitan monostearate. Additional surfactants that can be used include those that are generally characterized as Pluronic surfactants such as poloxamers, and Lutensol surfactants such as C₁₀ polyoxyethylene.

It is believed that anionic surfactants may be useful as part of the surfactant component. In general, it is expected that anionic surfactants have a greater tendency to cause irritation to skin tissue.

The antifungal composition can include an amount of surfactant component sufficient to provide the composition with a desired emulsion stability and sufficiently low viscosity without foaming. The amount of the surfactant component in the hand disinfecting composition, can be about 0.5 wt. % to about 6 wt. %, and can be about 1 wt. % to about 5 wt. %. It should be understood that the antifungal composition can be provided without any surfactant component.

The antifungal composition can contain a release agent to assist with the sustained release of the antifungal component over a prolonged period of time. Because most antifungal components are relatively hydrophobic, the presence of a release agent can be advantageous to assist with the sustained release of the antifungal component. Relatively hydrophobic components have a tendency to be bound or contained within the antifungal composition because of the presence of the skin bonding polymer component. The presence of a release agent such as a surfactant can help the antifungal component to release from the composition.

### pH Adjusting Agent

The hand disinfecting composition can include a pH adjusting agent to provide the hand disinfecting composition with a pH that helps stabilize the disinfectant active component. Exemplary pH adjusting agents that can be used include sodium hydroxide, potassium hydroxide, triethylolamine, and mixtures thereof.

The polymer component of the lotion may be at least in part responsible for reducing the irritability of the antifungal composition at low pH values. For example, it is believed that the polymer component may help reduce irritation of skin tissue. The antifungal composition can be provided without any pH modifier, if desired.

### Thickener

Thickeners that can be incorporated into the antifungal composition include those components that thicken or increase the viscosity of the antifungal composition so that the antifungal composition can be readily applied to skin. Thickeners that can be used in the antifungal composition include those components often referred to as viscosity controlling agents.

Exemplary thickeners or viscosity controlling agents that can be provided in the hand disinfecting composition include cellulose gum, alkane triols; acrylates; substituted celluloses such as hydroxy ethyl cellulose, carboxymethyl cellulose, methylcellulose, and hydroxypropyl cellulose; cetyl alcohol; gums such as natural gums or synthetic gums; long chain alcohols such as those having about 9 to about 24 carbon atoms; polyglycols such as polyethylene glycols, polypropylene glycols, polybutylene glycols, polyethylene propylene glycols, or mixtures thereof; waxes such as natural waxes or synthetic waxes; hydrogenated oils; glycol esters; fatty acid esters; long chain acids; acid amides; silicates; and mixtures thereof. An exemplary thickener that can be used is hydroxyethyl cellulose.

The antifungal composition may or may not include a thickener. When the antifungal composition includes a thickener, the thickener can be provided in an amount that provides the desired level of thickening. The hand disinfecting composition can include a thickener in an amount of least about 0.1 wt. % and can include a thickener in an amount of at least about 0.4 wt. %. In addition, the thickener can be provided in an amount of less than about 2 wt. %, and can be provided in an amount of less than about 1.0 wt. %. The antifungal composition can be provided without any thickener, if desired.

### Emollient

The antifungal composition can include an emollient for improving the texture of the composition. An emollient is an oleaginous or oily substance which helps to smooth and soften the skin, and may also reduce its roughness, cracking or irritation. Exemplary suitable emollients include mineral oil, having a viscosity in the range of 50 to 500 centipoise (cps), lanolin oil, coconut oil, cocoa butter, olive oil, almond oil, macadamia nut oil, synthetic jojoba oils, natural sonora jojoba oils, safflower oil, corn oil, liquid lanolin, aloe vera, cottonseed oil, and peanut oil.

Other suitable emollients include squalane, castor oil, polybutene, odorless mineral spirits, sweet almond oil, avocado oil, clophyllum oil, ricin oil, vitamin E acetate, olive oil, linolenic alcohol, coconut oil, oleyl alcohol, the oil of cereal germs such as the oil of wheat germ, isopropyl palmitate, octyl palmitate which is commercially available as Lexol EHP, tradename of Inolex Co. of Philadelphia, Pa., isopropyl myristate, hexadecyl stearate, butyl stearate, decyl oleate, acetyl glycerides, the octanoates and benzoates of (C₁₂-C₁₅) alcohols, the octanoates and decanoates of alcohols and polyalcohols such as those of glycol and glycerol, ricin oleates of alcohols and poly alcohols such as those of isopropyl adipate, hexyl laurate and octyl dodecanoate.

Other suitable emollients which are solids or semi-solids at room or ambient temperatures may be used in amounts sufficient to provide liquid topical compositions. Such solid or semi-solid cosmetic emollients include hydrogenated lanolin, hydroxylated lanolin, acetylated lanolin, petrolatum, isopropyl lanolate, butyl myristate, cetyl myristate, myristyl myrislate, myristyl lactate, cetyl alcohol, isostearyl alcohol and isocetyl lanolate. Exemplary emollients include stearic acid, stearyl alcohol, palmitic acid esters, natural and synthetic esters such as coconut oil.

The hand disinfecting composition can include the emollient in an amount sufficient to provide a silky feel. An exemplary range of the emollient in the hand disinfecting composition can be at least about 0.5 wt. %. In addition, the hand disinfecting composition can include an emollient in an amount of less than about 3 wt. %. It should be understood that the emollient is an optional component of the antifungal composition. The antifungal composition can be provided without an emollient, if desired.

### Moisturizer

The antifungal composition can include a moisturizer to provide a desired moisturizing effect to skin tissue. The moisturizer can be provided as a humectant. In general, a humectant is a moistening agent that promotes retention of water due to its hydroscopic properties. Exemplary humectants include glycerine, polymeric glycols such as polyethylene glycol and polypropylene glycol, and sorbitols such as sorbitol solution, pyrrolidone carboxylic acid, urea, or mixtures thereof. The hand disinfecting composition can be provided without a moisturizer.

When the antifungal composition includes a moisturizer, it can be included in an amount of at least about 0.5 wt. %. In addition, the antifungal composition can include a moisturizer in an amount of less than about 5 wt. %. It should be understood that the antifungal composition can be provided without a moisturizer.

### Preservatives

The antifungal composition can include preservatives for prevention of bacterial, fungal, and/or yeast contamination. Exemplary preservatives that can be used in the hand disinfecting composition include phenoxyethanol, benzoic acid, derivatives and salts of benzoic acid, parabens, oxazolidines, chlorinated aromatic compounds and phenols, hydantoins, cresols and derivatives, imiazolindinyl urea, iodopropanol butylcarbamate, sulfites, and bisulfites. The antifungal composition can include any of the preservatives commonly used or known to be suitable for topically applied compositions. Exemplary commercially available preservatives include liquid Germal Plus (diazolidinyl urea and iodopropynyl butylcarbanate) and Germaben 11 (diazolidinyl urea and methylparaben and propylparaben).

The antifungal composition can be formulated without a preservative. It is expected that the preservative will increase the shelf life of the antifungal composition by reducing or preventing the growth of bacteria, fungus, and/or yeast. When the antifungal composition includes a preservative, the preservative is preferably provided in an amount sufficient to provide a desired level of protection from growth of bacteria, fungus, and/or yeast.

In general, for most preservatives, it is expected that the amount of preservative can be provided at a level of about 0.1 wt. % to about 1.0 wt. %, and can be provided at a level of about 0.2 wt. % to about 0.5 wt. %, based on the weight of the antifungal composition. Because of the relatively large amount of the disinfectant active component in the antifungal composition, the antifungal composition can be provided without a supplemental preservative.

### Antioxidants

The hand disinfecting composition can include antioxidants to help increase the shelf life of the antifungal composition. Exemplary antioxidants that can be used include vitamins such as vitamin E, vitamin E acetate, vitamin C, vitamin A, and vitamin D, and derivatives thereof. Exemplary antioxidants include α-tocopherols which can be characterized as natural or synthetic Vitamin E. Additional exemplary antioxidants include propyl, octyl and dodecyl esters of gallic acid, butylated hydroxyanisole (BHA) (usually as a mixture of ortho and meta isomers), butylated hydroxytoluene (BHT), and nordihydroguaiaretic acid, and alkylated parabens such as methylparaben and propylparaben. Another type of antioxidant includes a reducing component such as a reducing sugar to stabilize the disinfectant active component. And exemplary reducing sugar includes glucose.

The antifungal composition can be formulated without an antioxidant. When the hand disinfecting composition includes an antioxidant, the antioxidant can be provided in an amount that provides antioxidant properties in the antifungal composition. In general, it is expect that the antioxidant can be provided in an amount of about 0.2 wt. % to about 2 wt. %, and can be provided in an amount of about 0.7 wt. % to about 1.5 wt. %, based on the weight of the hand disinfecting composition. In the case of vitamin E, it is expected that the vitamin E can be included in the antifungal composition in an amount of about 0.1 wt. % to about 1 wt. %, and can be included in an amount of about 0.3 wt. % to about 0.8 wt. %. It should be appreciated that the hand disinfecting composition can be provided without an antioxidant.

### Chelating Agents

Chelating agents are substances used to chelate or bind metallic ions with a certain heterocyclic ring structure so that the ion is held by chemical bonds from each of the participating rings. Suitable chelating agents include ethylene diaminetetraacetic acid (EDTA), EDTA trisodium, EDTA tetrasodium, calcium disodium edetate, EDTA trisodium, EDTA tetrasodium and EDTA dipotassium. One or more chelating agents can optionally be included in the emulsion in amounts ranging from about 0.001 to about 0.1 weight percent. It should be appreciated that the antifungal composition can be provided without a chelating agent.

### Fragrances

Fragrances are aromatic compounds which can impart an aesthetically pleasing aroma to the antifungal composition. Typical fragrances include aromatic materials extracted from botanical sources (i.e. rose petals, gardenia blossoms, jasmine flowers, etc.) which can be used alone or in any combination to create essential oils. Alternatively, alcoholic extracts may be prepared for compounding fragrances. One or more fragrances can optionally be included in the composition in an amount ranging from about 0.001 to about 10 weight percent, preferably about 0.05 to about 5 percent. It should be appreciated that the antifungal composition can be provided without a fragrance.

### Carriers, Diluents, and Excipients

The antifungal composition may also include non-toxic, pharmaceutically and dermatologically acceptable carriers, diluents and excipients, suitable for topical application, as are well known, see for example Merck Index, Merck & Co., Rahway, N.J., Bioreversible Carriers in Drug Design, Theory and Application, Roche (ed.) Pergamon Press, (1987), Gilman et al., (eds) (1990) Goodman and Gilman's: The Pharmacological Bases of Therapeutics, 8th Ed., Pergamon Press, Novel Drug Delivery Systems, 2nd Ed., Norris (ed.) Marcel Dekker Inc., (1989), and Remington's Pharmaceutical Sciences. For standard dosages of conventional pharmacological agents, see, e.g., Physicians Desk Reference (1997 Edition); and American Medical Association (1997) Drug Evaluations (Subscriptions).

An exemplary antifungal composition can be provided according to Table 1.

TABLE 1
Antifungal Treatment Composition

| Component | Amount (wt. %) |
|---|---|
| skin binding polymer component | 3-20 |
| antifungal active component | 0.1-3 |
| release agent | 0.5-6 |
| thickener | 0.1-2 |
| emollient | 0.5-3 |
| humectant | 0.5-5 |
| preservative | 0.1-1 |
| pH neutralizing agent | to pH of 4-8 |
| water | Balance |

### Example 1

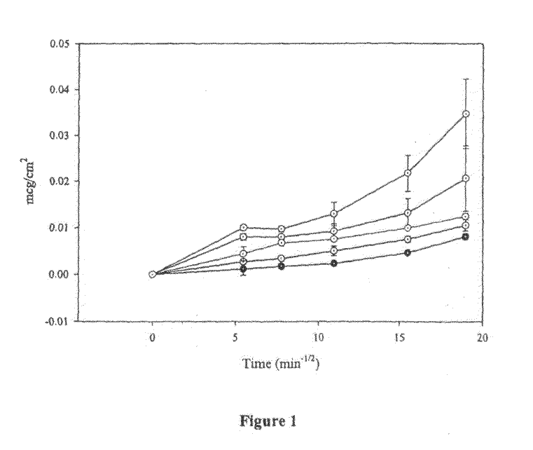

Results of a bioavailability test in Franz Cells for a composition having varying amounts of surfactant (Pluronic L-44 NF) is reported in FIG. 1. The composition contains 8 wt. % skin binding polymer component based on starch and poly(vinylpyrrolidone/alkylene), 1 wt. % naftifine, and water. The concentrations of surfactant range from 0 wt. % (lowest), 0.2 wt. %, 0.4 wt. %, 0.6 wt. %, and 0.8 wt. %. Increased amounts of surfactant relate to higher bioavailablity of naftifine.

### Example 2

The release of 1% terbinafine hydrochloride from a composition containing 8 wt. % skin bounding polymer prepared from cellulose gum and poly(vinylpyrrolidone/alkylene), 0.7 wt. % pluronic L-44 surfactant, and 1 wt. % terbinafine hydrochloride is compared with release of 1 wt. % terbinafine hydrochloride from an over the counter lamisil product containing 1 wt. % terbinafine hydrochloride. As shown in FIG. 1, the lower curve represents the over the counter product.

We claim:

1. An antifungal composition comprising:
(a) an effective amount of an antifungal component to provide antifungal properties to skin tissue;
(b) an effective amount of a skin bonding polymer component to provide a sustained release of the antifungal component over a prolonged period of time after application of the antifungal treatment composition to skin tissue; and
(c) water.

2. An antifungal composition according to claim 1, wherein the antifungal component comprises at least one of clotrimazole, naftifine, ketoconazole, tolnaftate, terbinafine, butenafine, miconazole, metronidazole, fluconazole, and mixtures thereof.

3. An antifungal composition according to claim 1, wherein the composition provides sustained release of the antifungal component for at least about one hour after application of the composition to skin tissue.

4. An antifungal composition according to claim 1, wherein the composition comprises about 0.1 wt. % to about 3 wt. % of the antifungal component.

5. An antifungal composition according to claim 1, wherein in the skin bonding polymer component comprises a hydrophobic polymer/hydrophilic polymer adduct.

6. An antifungal composition according to claim 5, wherein in the hydrophobic polymer/hydrophilic polymer adduct comprises a poly (vinylpyrrolidone-alkylene) polymer and a polymer comprising repeating carboxylic acid groups hydroxyl groups, or a mixture of carboxylic acid groups and hydroxyl groups.

7. An antifungal composition according to claim 5, wherein the poly (vinylpyrrolidone-alkylene) polymer comprises a polymer having an alkylene group containing about 10 carbon atoms to about 30 carbon atoms.

8. An antifungal composition according to claim 1, wherein the skin bonding polymer component has a ratio of pyrrolidone groups to a combination of carboxylic acid groups and hydroxyl groups of about 1:1 to about 5:1.

9. An antifungal composition according to claim 1, wherein the composition comprises at least about 50 wt. % water.

10. An antifungal composition according to claim 1, wherein the composition comprises about 0.5 wt. % to about 6 wt. % of a surfactant.

11. An antifungal composition according to claim 1, wherein the composition comprises about 0.1 wt. % to about 2 wt. % of a thickener.

12. An antifungal composition according to claim 1, wherein the composition comprises about 0.5 wt. % to about 5 wt. % of a moisturizer.

13. An antifungal composition according to claim 1, wherein the composition provides a release rate of antifungal component after application to skin tissue at one hour that is within about 50% or the release rate at 30 minutes.

14. A method for using an antifungal composition comprising:
applying the antifungal composition to skin tissue, wherein the antifungal composition comprises:
(a) an effective amount of an antifungal component to provide antifungal properties to skin tissue;
(b) an effective amount of a skin bonding polymer component to provide a sustained release of the antifungal component over a prolonged period of time after application of the antifungal treatment composition to skin tissue; and
(c) water.
